(19) United States
(12) Patent Application Publication
(10) Pub. No.: US 2019/0177623 A1
(43) Pub. Date: Jun. 13, 2019

(54) Acetylene Production By Staged Combustion With Accommodative Cross-Sectional Area

(71) Applicant: SABIC GLOBAL TECHNOLOGIES B.V., Bergen op Zoom (NL)

(72) Inventors: Subramanian SANKARAN, Houston, TX (US); Balamurali Krishna RAMACHANDRAN NAIR, Sugar Land, TX (US); Sreekanth PANNALA, Sugar Land, TX (US); Pankaj Singh GAUTAM, Evansville, IN (US); Chunliang WU, Katy, TX (US)

(73) Assignee: SABIC Global Technologies B.V., Bergen op Zoom (NL)

(21) Appl. No.: 16/302,720

(22) PCT Filed: May 11, 2017
(86) PCT No.: PCT/US2017/032164

§ 371 Date: Nov. 19, 2018

Related U.S. Application Data
(60) Provisional application No. 62/341,868, filed on May 26, 2016.

(51) Int. Cl. C10G 9/38; C07C 2/78; C07C 4/02; B01J 19/26; F23C 6/04; F23C 3/00

(52) U.S. Cl. CPC C10G 9/38; C07C 2/78; C07C 4/025; B01J 19/26; F23C 6/047; F23C 2201/301; C10G 2400/24; C10G 2300/1081; C10G 2300/1088; C10G 2300/1025; F23C 3/00

(57) ABSTRACT

A systems and method for production of acetylene by pyrolysing a feedstock through combustion products are disclosed. The system comprises: a combustion chamber having a chamber structure including sidewalls; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and a process feed for providing a feedstock acted upon by the combustion within the combustion chamber, wherein a firing rate of about 30 MMBtu/h to about 1000 MMBtu/h is exhibited in the combustion chamber.

### TECHNICAL FIELD

The present disclosure generally concerns fuel combustors, and more specifically, fuel combustors including a combination of novel features to increase production.

### BACKGROUND

Production of chemicals can be achieved using a variety of processes. Using some techniques, heated chambers such as combustion chambers can be employed to combust various fuels in the production of particular chemicals. For example, fossil-based feedstock such as natural gas can undergo pyrolysis in a chamber to such ends. Burners (e.g., oxy-fuel burners) or jets in the chamber can be fired to provide this result.

For example, natural gas pyrolysis can be used to produce acetylene which is a chemical intermediate for various commodity chemicals such as ethylene. However, current pyrolysis reactors cannot utilize natural gas to produce ethylene with improved scalability and volume.

These and other shortcomings are addressed by aspects of the present disclosure.

### SUMMARY

In an aspect, a system is disclosed comprising: a combustion chamber having a chamber structure including sidewalls; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and a process feed for providing a feedstock acted upon by the combustion within the combustion chamber, wherein a firing rate of about 30 million (or thousand thousand) British Thermal units per hour (MMBtu/h) to about 1000 MMBtu/h is exhibited in the combustion chamber.

In some aspects, there can also be a method for producing a chemical comprising: firing a first jet within a combustion chamber at a first jet angle, the first jet angle defined by one or more first inlets through a sidewall of the combustion chamber, the first jet is fired by providing fluid through the one or more first inlets, the first jet is an axial jet; and firing two or more second jets within a chamber at two or more second jet angles, the two or more second jet angles defined by two or more second inlets through the sidewall of the combustion chamber, the two or more second jets are fired by providing fluid through the one or more second inlets, the two or more second jets are radial jets; and providing feedstock into the combustion chamber during combustion through a process feed, the feedstock is processed into at least a portion of a product output from the combustion chamber, wherein a firing rate of about 30 MMBtu/h to about 1000 MMBtu/h is exhibited in the combustion chamber.

Additional aspects herein include a system comprising a chamber having a chamber structure including sidewalls. The sidewalls can have two or more sections of varying cross-section. The system also includes a first stage having first inlets which have first inlet directions incident to respective areas of the sidewalls at first inlet angles. There are also two or more secondary stages having secondary inlets which have secondary inlet directions incident to respective areas of the sidewalls at secondary inlet angles. The secondary inlet directions vary by stage. There is also included a cooling structure about at least a portion of the chamber.

These and other aspects are described in greater detail elsewhere herein.

### BRIEF DESCRIPTION OF THE DRAWINGS

To better understand and appreciate disclosures herein, refer to the Detailed Description hereafter in connection with the accompanying drawings:

FIGS. 1A to 1C illustrate an example chamber described herein;

FIGS. 2A to 2G illustrate various examples of inlets and related bores used with some aspects of example chambers described herein;

FIGS. 3A to 3C illustrate an example chamber herein;

FIGS. 4A to 4C illustrate another example chamber herein;

FIGS. 5A to 5C illustrate other example chambers herein;

FIG. 6 illustrates another example chamber herein;

FIG. 7 illustrates another example chamber herein;

FIG. 8 illustrates another example chamber herein;

FIG. 9 illustrates another example chamber herein;

FIG. 10 illustrates another example chamber herein;

FIG. 11 illustrates another example chamber herein;

FIG. 12 illustrates another example chamber herein;

FIG. 13 illustrates another example chamber herein;

FIG. 14 illustrates another example chamber herein; and

FIG. 15 illustrates another example chamber herein.

### DETAILED DESCRIPTION

The present disclosure generally concerns accommodative combustion chambers supporting new firing rates for the production of chemicals. These accommodative chambers can include varying cross-sections, and a variety of stages of inlets for providing fuel in which the inlets can have varying directions.

Oxy-fuel combustors or burners can be used for chemical production processes such as hydrocarbon cracking which breaks the bonds of longer carbon chains resulting in molecularly simpler output chemicals. The combustion undertaken in such processes generates substantial heat and pressure which can be both necessary to the process and challenging to contain, and chamber technology related to such techniques remains in development.

The management of heat and pressure may be conducted according to particular production parameters, which can in some aspects necessitate consistent heat throughout the chamber and/or particular gradients of heat throughout. Fluid and thermal parameters for such production can be controlled, at least in part, based on combustion chamber geometry and the location, orientation, and strength (e.g., firing rate, pressure, fluid velocity) of burners (or individual jets or inlets) for fuel or other materials into the combustion chamber. In an example, a chamber can have at least one conical section, a first jet (which can be, e.g., an axial jet feeding a burner) and at least a second jet (which can be, e.g., a radial jet feeding a burner) to provide scalability and control. The at least one conical section can be used to control the relative velocity in specific volumes of the chamber, which increases jet stability by reducing the likelihood of blowout, and accommodate increased flow in wider areas where at least the second jet can be located.

To fuel the combustion, fluid fuel and/or oxygen can be provided through inlets. One or more of the inlets may feed a jet. In turn, one or more jets may feed a burner with fluids. These fluids are ignited within the chamber and combust providing heat and pressure, and in some cases products or byproducts related to the chemical reactions on which chamber output depends, after being provided through inlets about the chamber interior.

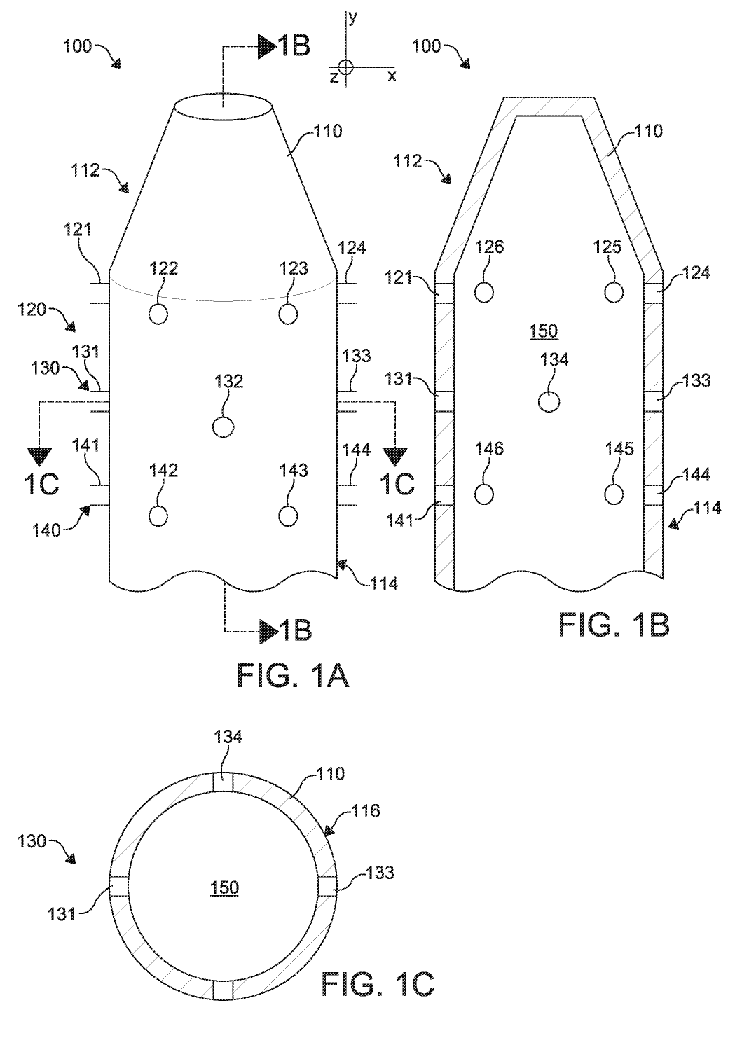

Combustion chambers described herein can be defined according to a coordinate system as illustrated in, e.g., FIG. 1, providing that the longest dimension of the chamber defines ay-axis which is complemented by convention x- and z-axes to provide relative directions with which to describe aspects of the chamber.

Chambers can also be described in terms of cross-sections, which can include any plane through the chamber, but will typically herein refer to a plane at a constant relative y-axis position extending through the chamber in x and z directions. On review of these disclosures, it will be understood that while chambers herein are drawn as having substantially annular sidewalls at such constant-y cross-sections, other possible arrangements having different chamber or cross-sectional shapes (e.g., oval, polygon) are contemplated and embraced under the disclosures herein. As used herein, the term “substantially,” in addition to its ordinary meaning, includes the meanings of completely, almost completely, to any significant degree, or to any acceptable limits or acceptable degree in accordance with those skilled in the art.

Chamber cross-section can vary based on the relative angular orientation of sidewalls, and chambers can accordingly be described in sections based on changes to cross-section. For example, a first section may include a tapered cross-section which enlarges to a subsequent cross-section which can be angled to taper at a different angle from the first cross-section or have a substantially constant cross-section. Chambers may include three or more sections including multiple sections which enlarge or reduce cross-sections throughout, to include both angled transitions which increase or reduce x and/or z dimensions over y or can be flat transitions increasing or decreasing other dimensions at a substantially constant y position. Further, changes to cross-sectional dimension can be effected using sidewalls having a curved contour along they-axis, providing, e.g., concave or convex sidewalls.

As suggested above, the fluid dynamics related to fluid flow, combustion, and pressure within the chamber can create a risk that burners/combustors will experience blowout or extinguishment during operation. Various chamber cross-sections can be useful to flame stability and may reduce coking, aiding in consistent flow and temperature fields, and decreasing the likelihood of a flame burning out. In one example, a conical section (or sections) can provide desired performance for improved flame stability and reduced coking. In such an example, recirculation-free “annular zones” have relatively lower velocities than core flow, improving flame stability for “swirl” flames therein. Increasing cross-section accommodates increased flow rates for additional “swirl” burners.

As used herein, an inlet is an opening through a solid surface (such as, e.g., a chamber sidewall) through which fluid can pass. Inlets have inlet directions which are the line about which the bore of the inlet is substantially centered as the inlet passes through the solid surface. In aspects, the bore may be non-constant (e.g., of changing bore cross-section in dimension or shape). In aspects, the inlet direction may vary through the solid surface. The inlet direction encounters the inner surface of the solid surface at an inlet angle, which defines the initial behavior the fluid exiting the inlet (along with any dynamics imparted the fluid during travel through the bore of the inlet or prior to entering the inlet, and subsequently the interaction of the fluid with other solid and fluid material such as chamber sidewalls and other fluids within the chamber).

One inlet or a group of inlet may operate to feed a burner for combustion. In certain aspects, one or more inlets may feed distinct jets, which may then cooperate to form a larger burner. As illustrated in the aspects herein, the burners of a respective stage can be substantially aligned at a particular position along the y-axis. As an example, a stage may be defined by a plurality of burners positioned in an annular configuration along a plane in the y-axis. However, stages of burners may include any number of burners and may be positioned in various configurations.

As described herein, a first stage can include a stage which produces an axial jet (and resultant flame) in a chamber. Further, as described herein, a second or stage can include a stage which produces a radial jet or flame in a chamber. Radial jets or flames may be “swirl” jets or flames. By using both first and second stages, firing rates can be achieved which exceed those possible using a single burner or multiple burners in a non-coordinated fashion. Inlets and/or Jets can be straight or curved (to include cylindrical), fine or wide, or have various other qualities or arrangements herein.

The stages may include additional geometry or components based on the particular parameters of a combustion chamber. For example, one or more burners may include an injector head. Injector heads or shaped inlets can be slotted or otherwise arranged to provide particular flow qualities or control over a resultant flame.

FIGS. 1A to 1C illustrate an example chamber 100 described herein. The chamber 100 includes sidewalls 110 comprised of at least first sidewall section 112 and second sidewall section 114. As shown, the first sidewall section 112 tapers from its largest dimension at its incidence with the second sidewall section 114 to a smallest dimension at a cap 116. The second sidewall section 114 is substantially constant in cross-section (save differences due to inlets of stages 120, 130, 140). This is just one possible example, and it is understood that other formations and dimensions may be utilized in alternative aspects, such as where the cap 116 can be larger than the cross-section of a subsequent sidewall section with which it is not in contact.

Exterior elements such as gas or fuel sources can reach chamber interior 150 using various inlets. The chamber 100 is illustrated with three stages of inlets. The first stage 120 includes inlets 121-126, the second stage 130 includes inlets 131-134, and the third stage 140 includes inlets 141-146. As discussed elsewhere, the inlets 121-126, 131-134, and 141-146 can be angled, shaped, or coupled with other components depending on fluid kinematics for the particular chamber and operational parameters. As described herein, the inlets 121-126, 131-134, and 141-146 may allow the passage of a fuel or oxygen, or both. Such fuel may undergo combustion to function as a burner. The configuration of the inlets 121-126, 131-134, and 141-146 relative to each other may be configured to generate particular thermal and fluid dynamics within the chamber 100. For example, each of the stages 120, 130, 140 may have inlets configured to direct flames in a given direction relative to the surface of the chamber 100.

The chamber 100 can be constructed of various materials. In an aspect, at least the internal portion of the chamber 100 (e.g., defining the chamber interior 150) can be constructed of a refractory-lined stainless steel. In alternative or complementary aspects portions of the chamber 100 can be constructed of ceramics or other materials, which can be tiled, cemented, et cetera. Various dimensions for one or more materials can be varied in terms of thickness, size, and so forth. These materials and material parameters provide structure and support which resists the heat and pressure of combustion performed in the chamber 100 during pyrolysis or other processes of chemical production.

A cooling structure (not shown in figure) can be provided around at least a portion of the chamber 100. The cooling structure can be a water jacket or various other structures in thermal communication with at least a portion of the chamber 100. These can be used to enlarge the serviceable life of the chamber 100 and/or maintain material temperatures during combustion operation for, e.g., pyrolysis.

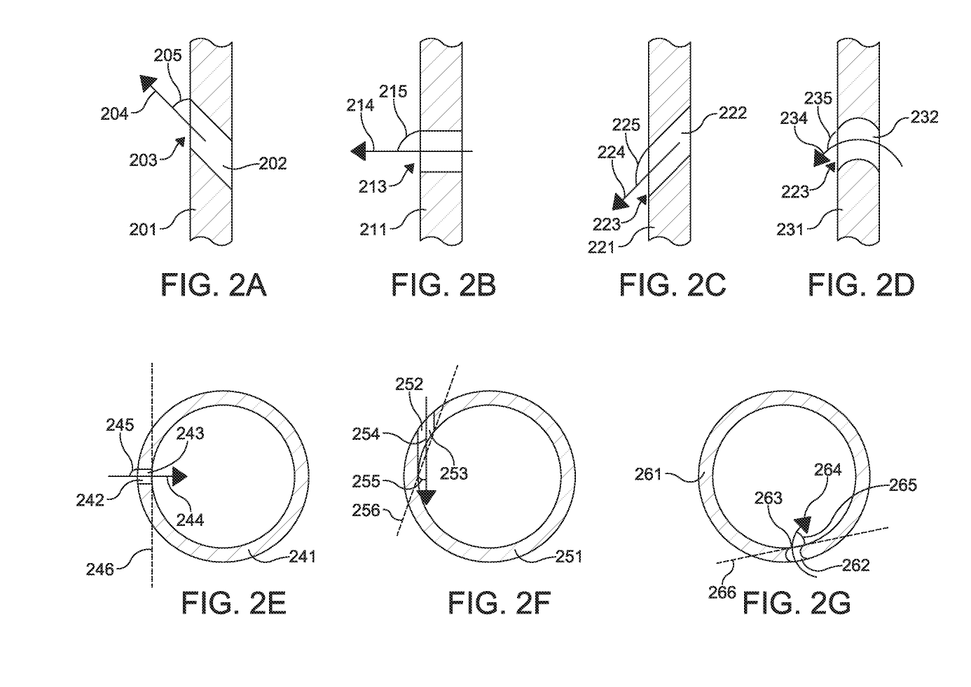

While the inlets of FIGS. 1A to 1C are shown substantially uniform in and through the chamber 100, various alternatives are contemplated herein. FIGS. 2A to 2G illustrate various examples of inlets and related bores used with some aspects of example chambers described herein. FIGS. 2A to 2D illustrate cutaway views in y-x or y-z planes, whereas FIGS. 2E to 2G illustrate cutaway views in x-z planes.

FIG. 2A illustrates inlet 203, which is angled through sidewall 201 relative to, e.g., faces of the sidewall 201 illustrated linearly in two dimensions. The sidewall 201 may be a sidewall of a chamber such as a combustion chamber. The inlet 203 may feed a burner configured to combust a material in the chamber. Other configurations are contemplated herein. It is understood that the drawings are two-dimensional representations of three-dimensional chambers, and accordingly angles will also be in three directions, and may include a range of possibilities within or vary from the angles illustrated. By angling inlets, fluids provided into a combustion chamber can be directed, dispersed, released, or otherwise provided according to the desired dynamics of uncombusted, combusting, and combusted fluids. As shown, bore 202 proceeds through sidewall 201 in a substantially linear manner and maintaining constant inlet direction 204 to establish inlet angle 205. This at least partially influences the fluid dynamics of fluid travelling into a chamber through inlet 203. As shown in FIG. 2A, inlet directions may be acute with respect to sidewall 201.

FIG. 2B illustrates inlet 213 of bore 212 through sidewall 211. The sidewall 211 may be a sidewall of a chamber such as a combustion chamber. The inlet 213 may feed a burner configured to combust a material in the chamber. Other configurations are contemplated herein. Inlet angle 215 can be substantially perpendicular to sidewall 201 based on inlet direction 214. Likewise, FIG. 2C illustrates inlet 223 of bore 222 through sidewall 221, having an obtuse inlet angle 225 based on inlet direction 224. The sidewall 221 may be a sidewall of a chamber such as a combustion chamber. The inlet 223 may feed a burner configured to combust a material in the chamber. Other configurations are contemplated herein.

FIG. 2D illustrates a bore 232 through sidewall 231, which in some aspects may be curved. The sidewall 231 may be a sidewall of a chamber such as a combustion chamber. The inlet 223 may feed a burner configured to combust a material in the chamber. Other configurations are contemplated herein. Inlet direction 234 curves with bore 232 and exits sidewall 231 at inlet angle 235.

FIG. 2E illustrates sidewalls 241 with bore 242 there through establishing inlet 243 and inlet direction 244. Inlet 243 has inlet angle 245, which can be measured from a tangent 246 of the sidewalls 241 at (e.g., the center of) bore 242. Accordingly, inlet angle 245 can be defined. Inlet angle 245 is substantially 90 degrees in the aspect shown in FIG. 2E.

FIG. 2F illustrates sidewalls 251 having bore 252 there through to establish inlet 253 and define inlet direction 254 and inlet angle 255 relative to tangent 256. As an example, the inlet 253 may be formed in the sidewall 251 of a jet configured to operate as a burner. As a further example, the inlet 253 may allow passage of a fuel, or oxygen, or both to pass into a cavity of a burner for combustion. Any number of the inlets 253 may be formed therein. A plurality of inlets 253 may be used together (with or without other inlets having other inlet angles) to generate a swirl flame.

FIG. 2G illustrates sidewalls 261 having curved bore 262 there through establishing inlet 263 and defining curved inlet direction 264. Inlet angle 265 can be measured in an instantaneous or terminal (e.g., final direction on passing beyond the inside of sidewalls 261) direction relative to tangent 266.

While FIGS. 2A to 2G illustrate various two-dimensional representations of bores creating inlets for chambers, it is understood that three-dimensional variation is embraced by the disclosures herein, and that combinations of the aspects of FIGS. 2A to 2D and FIGS. 2E to 2G can be realized without departing from the scope or spirit of the present disclosure. For example, a bore may be curved or angled through all three dimensions. Further, the curves and angles shown are merely for illustrative purposes, and can be any angle or curve through a sidewall.

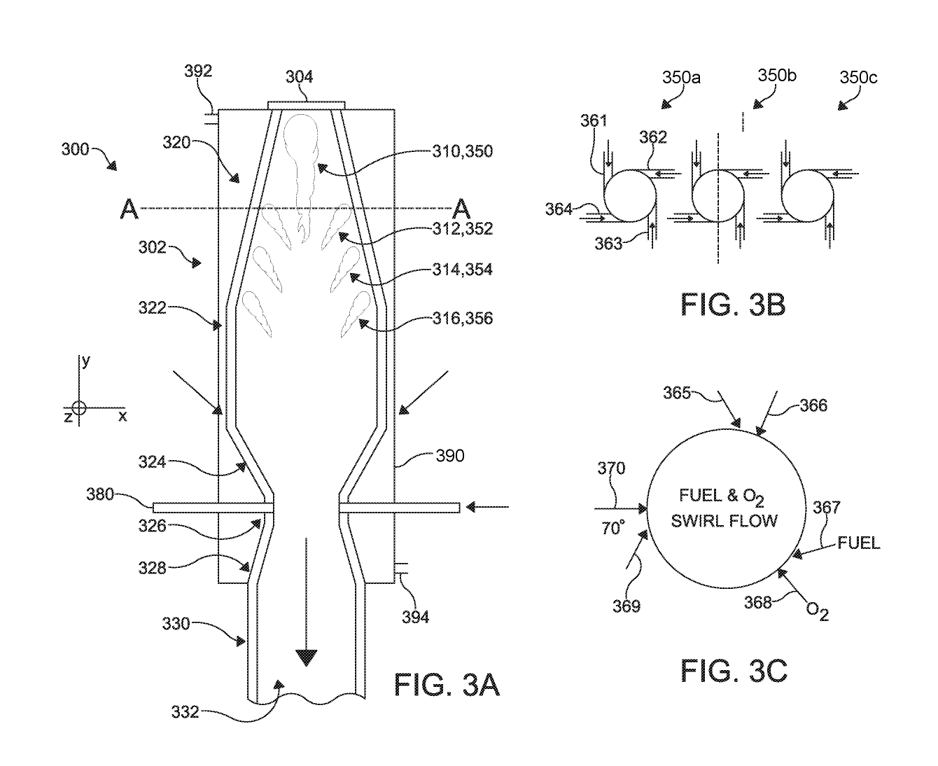

FIGS. 3A to 3C illustrate an example chamber 300 herein. The chamber 300 can be a combustion chamber used for processes such as pyrolysis in chemical production. The chamber 300 includes a first stage 310, a second stage 312, a third stage 314, and a fourth stage 316. However, any number of stages may be include and may be selectively operated. The first stage 310 may be configured to produce a first (e.g., axial) swirl jet (and resultant flame) while at least second, third, and fourth swirl jets (e.g., radial jets, diffusion flames) are produced by second stage 312, third stage 314, and fourth stage 316, respectively. Although the chamber 300 is illustrated having four stages 310, 312, 314, 316, any number of stages having any number of jets/burners arranged in any configuration to introduce thermal energy (e.g., flames) into the chamber 300. As an example, the chamber 300 (and other example chambers herein) may be illustrated in two dimensions, thereby illustrating jets/burners and stages along a plane of the chamber 300. However, arrangements of the jets/burners may include an annular arrangement of 2, 4, 6, or more burners along a horizontal plane of the chamber 300. Furthermore, the burners may be staggered along a longitudinal axis. Various arrangements may be used to control the thermal and fluid dynamics within the chamber 300, as is discussed in further detail below.

The first stage 310, second stage 312, third stage 314, and fourth stage 316 may each include inlets which provide fuel and/or oxygen for combustion in chamber 300. The inlet angles of the inlets of various stages can be varied to provide the desired geometries and fluid dynamics to support processes undertaken using chamber 300 both in terms of how the fluids enter the chamber as well as how they subsequently combust and interrelate with other fluids from other sources. Further, the inlets may feed an injector head, and the injector head may be angled to direct a flame in a particular direction relative to the chamber.

The chamber 300 has sidewall 302 and is illustrated with six sections. The sections are identified for description and are not intended to be limiting. From cap 304, a first section 320 expands conically to a second section 322, angled in a manner providing substantially constant cross-section. The second section 322 attaches opposingly to a third section 324, which narrows to a fourth section 326, which is mechanically coupled with process feed 380. The fourth section 326 is coupled with a fifth section 328, which is angled to increase the cross-sectional area of chamber 300. A sixth section 330 again changes the sidewall angle relative to its adjacent section, and provides an output 332 which either outputs a desired product or passes material to a subsequent production stage for additional processing.

The first stage 310 produces at least a first jet 350, the second stage 312 produces at least a second jet 352, the third stage 314 produces at least a third jet 354, and the fourth stage 316 produces at least a fourth jet 356. In aspects, first stage 310 may include a plurality of jets 350a, 350b, 350c configured to collectively function to generate an axial flame. For example, each jet 350a, 350b, 350c of the first stage 310 may be fed by first inlets 361, 362, 363, and 364, respectively, as shown in FIG. 3B. As a further example, the first inlets 361, 362, 363, and 364 may be configured to generate swirl jets. Each of the jets 350a, 350b, 350c may be configured to introduce fluids such as oxygen, fuel, or a combination into the chamber 300. The jets 350a and 350c may be configured to introduce oxygen, while the center jet 350b introduces fuel such as natural gas or methane. However, other fluids may be introduced such as a mix of natural gas and oxygen. By configuring the fluids passing through the first inlets 361, 362, 363, and 364, each of the jets 350a, 350b, 350c, may be controlled to produce a particular flame.

One or more of the second stage 312, third stage 314, and fourth stage 316 may include a radial jet 352, 354, 356 configured to be combusted to function as a burner. Any number of the radial jets 352, 354, 356 may be included in any stage. As an example, one or more of the radial jets 352, 354, 356 may be fed by inlets such as inlets 365, 366, 367, 368, 369, and 370 illustrated in FIG. 3C. As shown, the inlets 365, 366 and 369 may be oriented according to a first inlet angle, and the inlets 366, 368, and 370 may be oriented at a second inlet angle, creating complementary pairs of inlets. As will be set forth below, different stages and different inlets within stages can provide fluid at different flow rates from other inlets.

In aspects of chamber 300 or other chambers herein, stages can correspond to sidewall sections (e.g., one stage per section). However, in various aspects, a single sidewall section can include two or more stages or no stages at all.

The chamber 300 may include a cooling feature for managing the thermal energy at or near the sidewall 302 of the chamber 300. As shown, the chamber 300 may be at least partially surrounded by a cooling structure 390. The cooling structure 390 may be, e.g., a water jacket or other fluid-based coolant, and may include coolant inlet 392 and coolant outlet 394 to circulate a coolant fluid. Other cooling features such as heat exchangers may be used.

FIGS. 3B and 3C in particular illustrate sectional views looking down the y-axis. FIG. 3B shows inlets including non-mixed gases being injected into the chamber through the inlets. FIG. 3C shows different inlet angles and how angles can vary within individual stages to provide different geometries and dynamics for, e.g., oxygen and fuel flows.

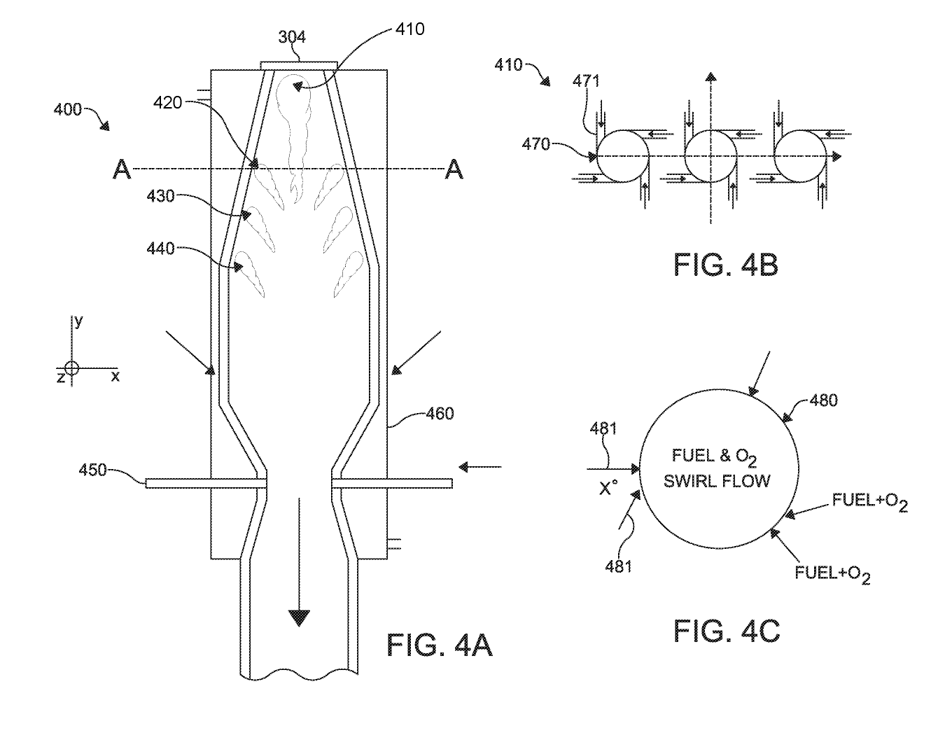

FIGS. 4A to 4C illustrates another example chamber 400. The chamber 400 employs pre-mixed gas jets with fuel and oxygen being provided mixed from inlets. FIG. 4A shows a chamber 400 having first stage 410, second stage 420, third stage 430, and fourth stage 440. The chamber 400 may be at least partially surrounded by a cooling structure 460, which, in aspects employing coolant fluid, includes a coolant inlet and coolant outlet. A process feed 450 provides chemicals for processing using at least the combustion gas in chamber 400.

The first stage 410 may be configured as an axial burner to generate a flame that extends generally axially related to a longitudinal axis of the chamber 400. As an example, the first stage 410 may include one or more burners or jets 470 fed by one or more inlets 471. FIG. 4B shows three jets 470, wherein each jet 470 is configured to be fed pre-mixed gases from four inlets 471. Such mixed gas may include natural gas and oxygen. Gases provided may have various ratios.

Returning to FIG. 4A, the second stage 420, third stage 430, and fourth stage 440 may be configured as a radial burner to generate a flame that extends generally radially related to a longitudinal axis of the chamber 400. As shown, each of the radial burners included in the respective one of the stages 430, 430, 440 is angled away from the first stage 410 such that a flame is non-orthogonal to a longitudinal axis of the chamber 400. Various angles of the radial burners in each stage 420, 430, 440 may be used to effect various conditions within the chamber 400. As an example, each of the second stage 420, third stage 430, and fourth stage 440 may include one or more burners or jets 480 fed by one or more inlets 481. FIG. 4C shows an example jet 480 configured to be fed pre-mixed gases from six inlets 481. Such mixed gas may include natural gas and oxygen. Gases provided may have various ratios. As shown, a pair of the inlets 481 may have an inlet angle (X°) relative to each other. Configuration of the inlet angles may be used to create swirl flame at one or more of the jets 480.

Performance of the aspects of FIGS. 3 and 4, and other aspects herein, can be influenced by, e.g., fuel, oxygen, and capacity. In specific aspects, fuel can include million standard cubic feet of gas per day (MM SCFD), O₂ can include 26 MM SCFD, and ethylene capacity can be 50 kiloTons per annum (kTA).

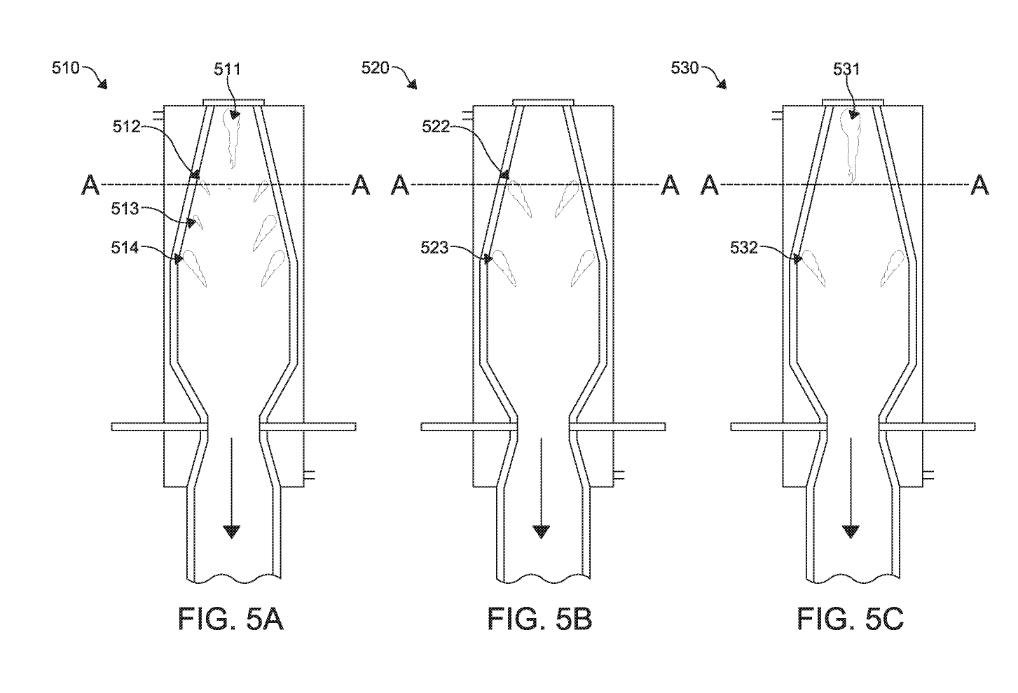

FIGS. 5A to 5C illustrate other example chambers 510, 520, and 530, emphasizing control of flow rate for both axial and radial burners/jets to provide specific characteristics according to various operational parameters. Particularly, in FIG. 5A, the chamber 510 shows an axial jet 511 with controlled flow, a first radial stage 512 having one or more radial jets, a second radial stage 513 having one or more radial jets with restricted flows, and a third radial stage 514 having one or more radial jets with less-restricted flow relative to the second stage 513. In this way, a temperature gradient may be leveled or managed in a pyrolysis process where the axial jet 511 and the third radial stage 514 provide greater energy to the system than the first radial stage 512 and the second radial stage 513, which can provide sufficient energy to establish or maintain a desired temperature while reducing the likelihood of temperature drops between the higher-output jets or risking overheating. Although each radial stage 512, 513, 514 is shown having two jets/burners, such an illustration is for example only. Nay number of jets/burners may be included in each stage.

With reference to FIG. 5B, the chamber 520 shows a first radial stage 522 and a second radial stage 523 having one or more axial jets. As shown, any axial burners/jets may be completely restricted (if present). In this manner, temperature can be focused within certain portions of the chamber 520, rate of heating can be controlled as the chamber 520 proceeds to processing temperature, temperature gradients can be established, fluid dynamics within the chamber 520 can be controlled and so forth.

With reference to FIG. 5C, the chamber 530 shows an axial stage 531 having axial jets with greater flow than that in, e.g., the chamber 510, and one radial jet 532. As described above, such a configuration can provide additional control over temperatures for particular processing parameters and manage fluid dynamics that might otherwise create unwanted or unpredictable interactions between jets (e.g., blowout). These are but a few possible examples of combinations of different numbers and orientations of axial and radial jets with flow control capability by stage and/or inlet. Controlled flow can be accomplished by, e.g., increasing or decreasing pressurization of gases provided, mechanically restricting or opening the inlets, bores, or external interfaces thereto, and by other techniques.

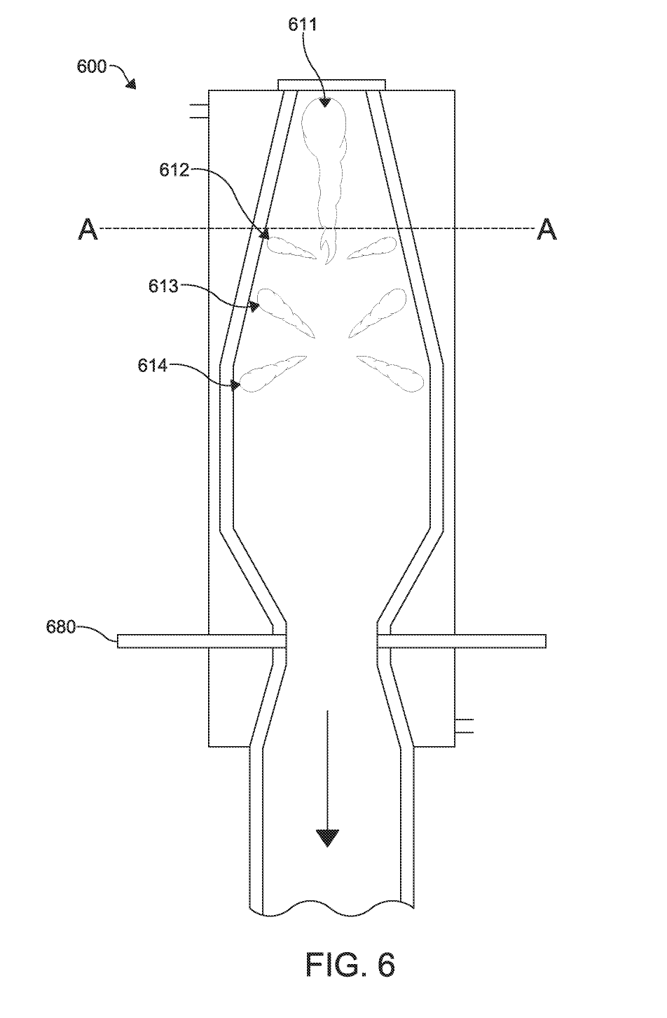

FIG. 6 illustrates another example chamber 600 herein which includes an axial stage 611 having one or more jets, a first radial stage 612, a second radial stage 613, and a third radial stage 614, where each radial stage 612, 613, 614 comprises radial jets have varying inlet angles by stage. For example, the jets of the first radial stage 612 may be angled generally orthogonal to a longitudinal axis of the chamber 600. As another example, the jets of the second radial stage 613 may be angled away from the axial stage 611. As a further example, the third radial stage 614 may be angled toward the axial stage 611. Such a configuration can assist with, e.g., mixing of the fluids including the chemical(s) provided through process feed 680 as well as providing for other considerations such as those given with regard to other example aspects. The angles of each jet in each stage may be varied to control the thermal and fluid dynamics in the chamber 600.

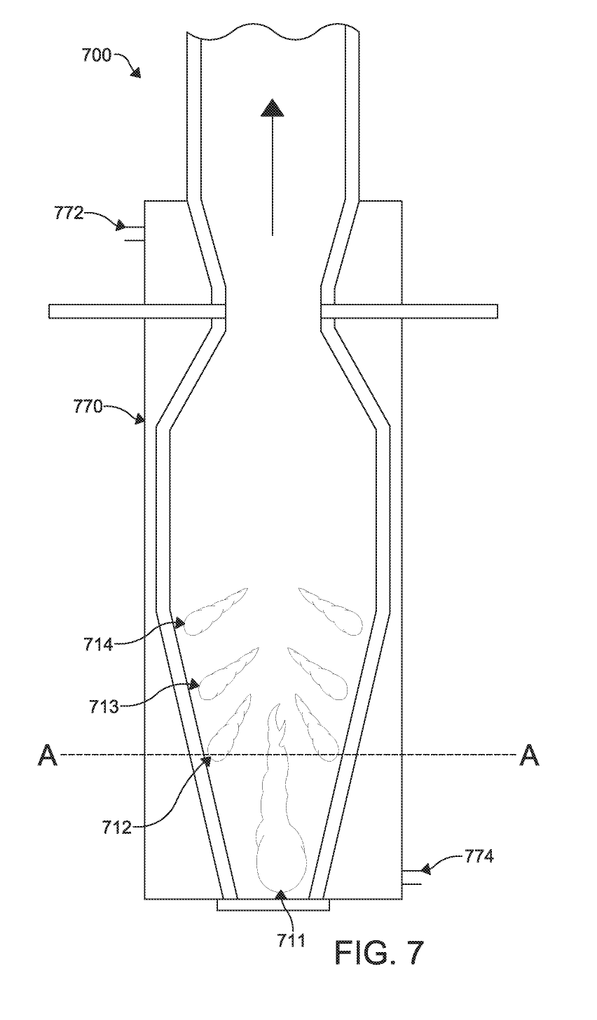

FIG. 7 illustrates another example chamber 700 herein. The chamber 700 is arranged with a different orientation than that shown elsewhere, facilitating alternative fluid dynamics, thermodynamics, and ultimately production qualities. FIG. 7 indicates only one possible orientation of the chamber 700 or other chambers herein, and others are embraced herein without limitation. As shown, the orientation of a cooling structure 770 may vary based on the orientation of the chamber 700, and so coolant in/out 772 and coolant in/out 774 may be swapped or interchangeable as compared to similar elements of alternative aspects. The chamber 700 includes inlets for producing axial jet 711, first radial stage 712, second radial stage 713, and third radial stages 714 having jets disposed at absolute or relative angles such as those shown or others.

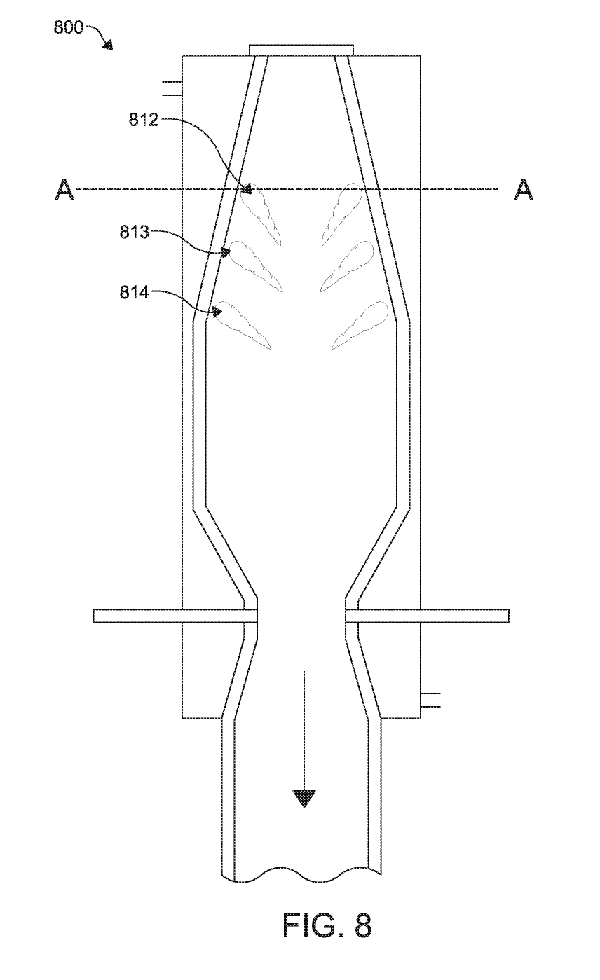

FIG. 8 illustrates another example chamber 800 herein providing another example without an axial burner/jet (e.g., no inlets for axial jet, or axial jet restricted to zero flow). The chamber 800 includes a first radial stage 812, second radial stage 813, and third radial stage 814, wherein each of the stages 812, 813, 814 may comprise one or more jets configured to provide differing thermal profiles (e.g., fuel rate, fuel ratio, etc.) at each respective stage 812, 813, 814. For example, less fuel pressure or fuel ratio may be provided to the jets of the first radial stage 812 than that provided to the jets of the third radial stage 814.

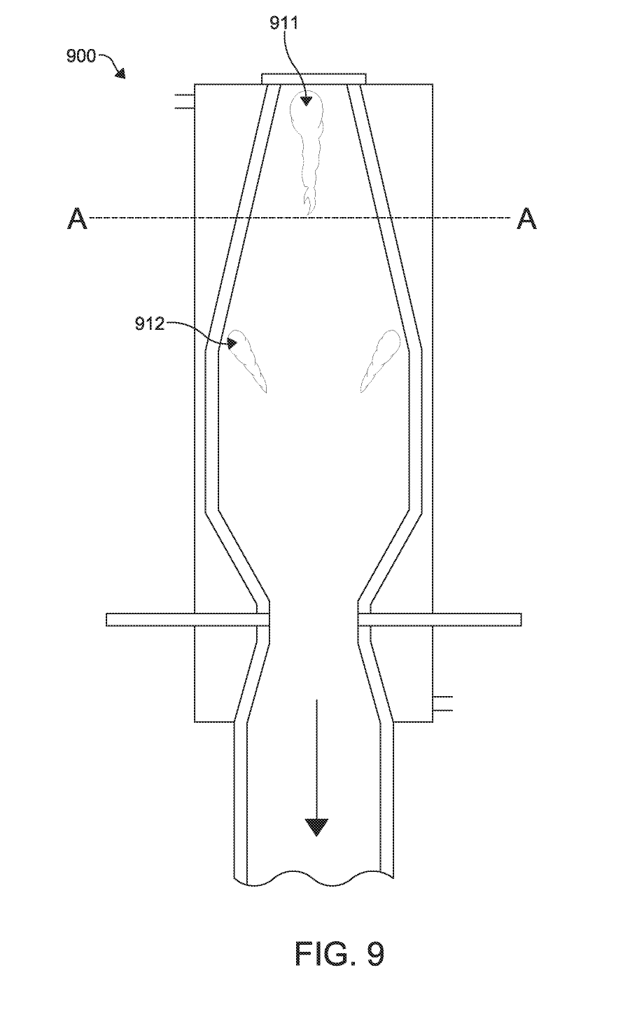

FIG. 9 illustrates another example chamber 900 herein. The chamber 900 can include a radial stage 912 having one or more jets and an optional/variable number of axial stages 911 having one or more jets. As shown, the axial stage 911 may comprise two or more axial jets produced from one or more inlets. The axial stage 911 may comprise a plurality of axial jets configured to introduce thermal energy in a portion of the chamber 900 that is spaced from the axial stage 911. Furthermore, the jets of the radial stage 912 may be angled to introduce a flame having an angle away from the axial stage 911. Such a configuration may facilitate a custom approach to managing the fluid and thermal dynamics of the chamber 900.

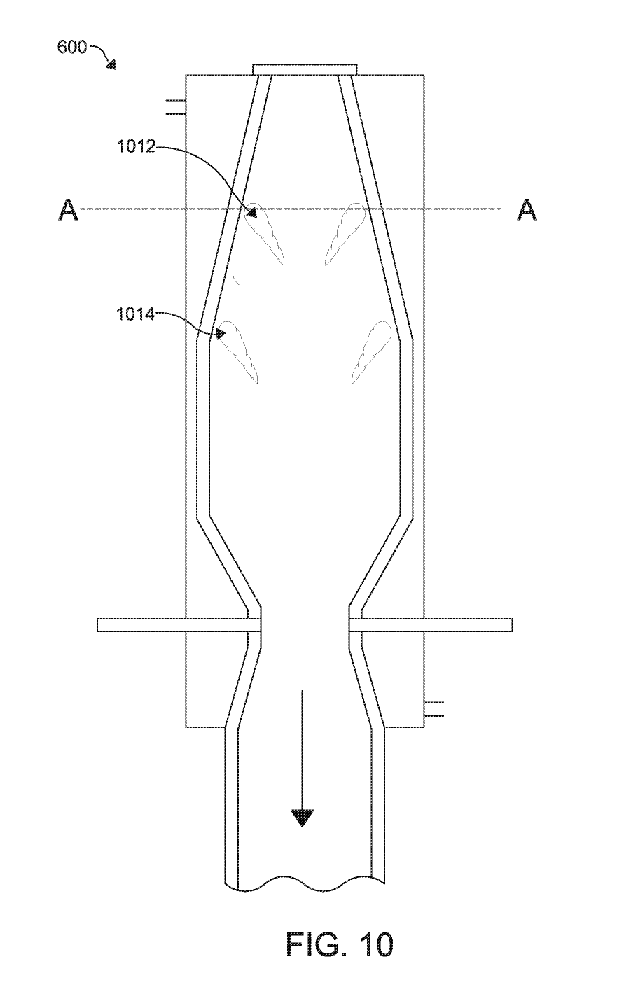

FIG. 10 illustrates another example chamber 1000 herein. Chamber 1000 can include a variable number of radial jets, with a first radial stage 1012 and a second radial stage 1014 each comprising one or more radial jets. As illustrated, each of the stages 1012, 1014 includes an annular arrangement of radial jets. However, other configurations of jets may be used. In alternative or complementary aspects any number of axial and/or radial flames can be designed into chambers disclosed herein.

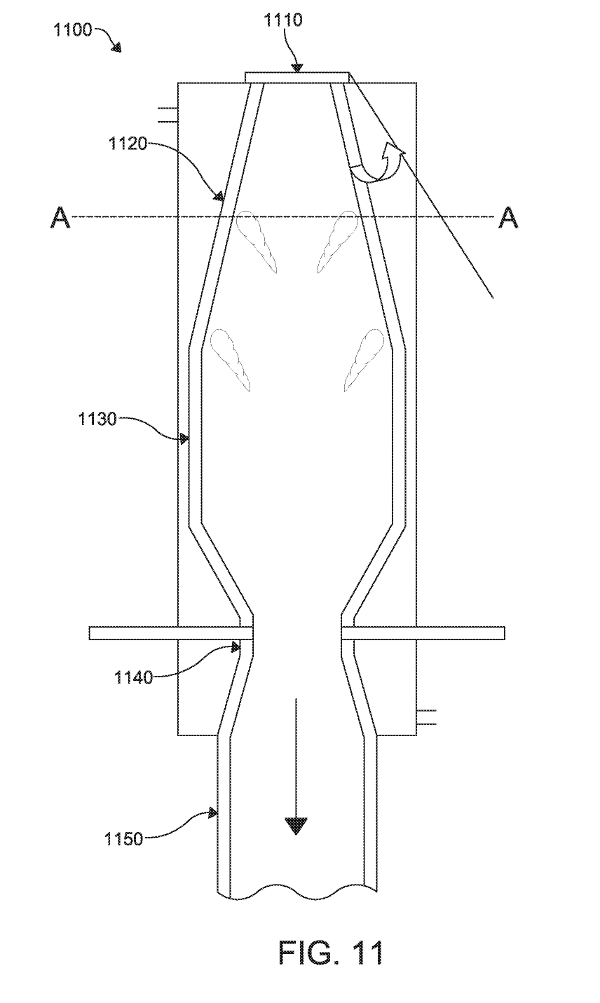

FIG. 11 illustrates another example chamber 1100 herein having a variable angle defining one or more of the chamber 1100 sections. Chamber 1100 can include a first section 1110, a second section 1120, a third section 1130, and feed section 1140 operatively coupled with output 1150. As illustrated, it is shown that first section 1110 can have a conical contour and that the angle of first section 1110 with respect to, e.g., an arbitrary axis, lateral axis, or second section 1120 can vary to make the conical arrangement wider or narrower.

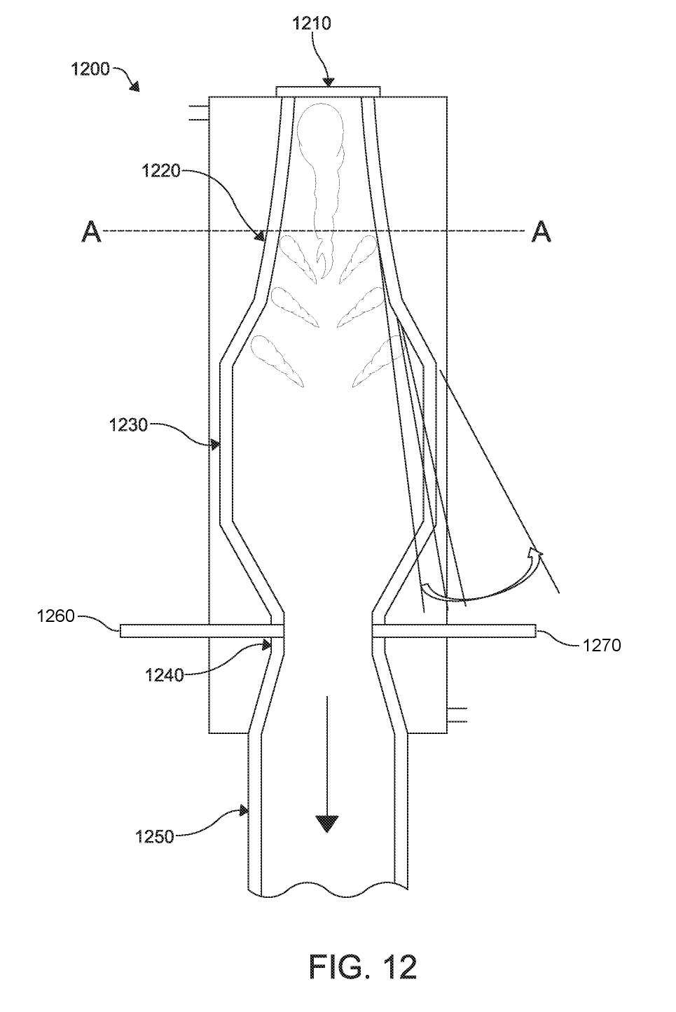

FIG. 12 illustrates another example chamber 1200 herein. Chamber 1200 can include multiple sections having different angles defining a variable conical contour. Such angular changes can accommodate alternative or additional flow rates from inlet stages to facilitate flow uniformity or desired dynamics. A stepped arrangement can also assist with flame stability and protect adjacent jets from blowout/extinguishment due to fluid interactions within the chamber. Specifically, chamber 1200 includes first section 1210, second section 1220, third section 1230, fourth section 1250, and feed section 1260 coupled to outlet 1270. Various cone angles relative to two or more of the first section 1210, second section 1220, third section 1230, fourth section 1250, feed section 1260, and outlet 1270 may be configured based upon burn rate and or other factors.

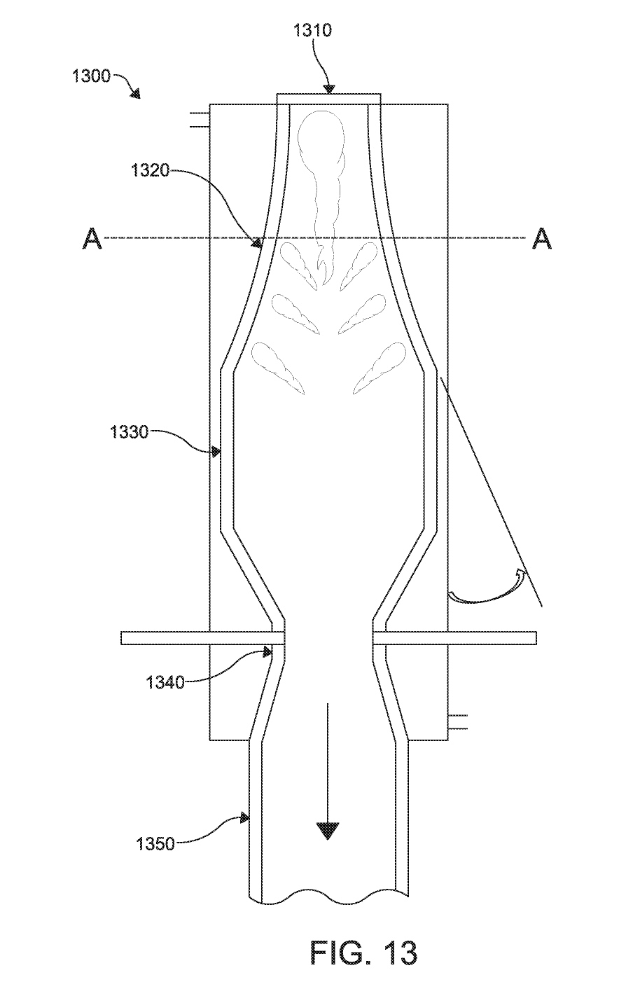

Further, elements of aspects herein need not be constrained to cylindrical or “linear”-conical arrangements. FIG. 13 illustrates another example chamber 1300 herein including surfaces curved along the y-axis in the conical section. Particularly, chamber 1300 includes first section 1310, second section 1320, third section 1330, and mixing section 1340 coupled to output 1350. The second section 1320 is a “non-linear”-conical section having a progressive or curved contour, and can include one or more axial jet stages and/or one or more radial jet stages therein.

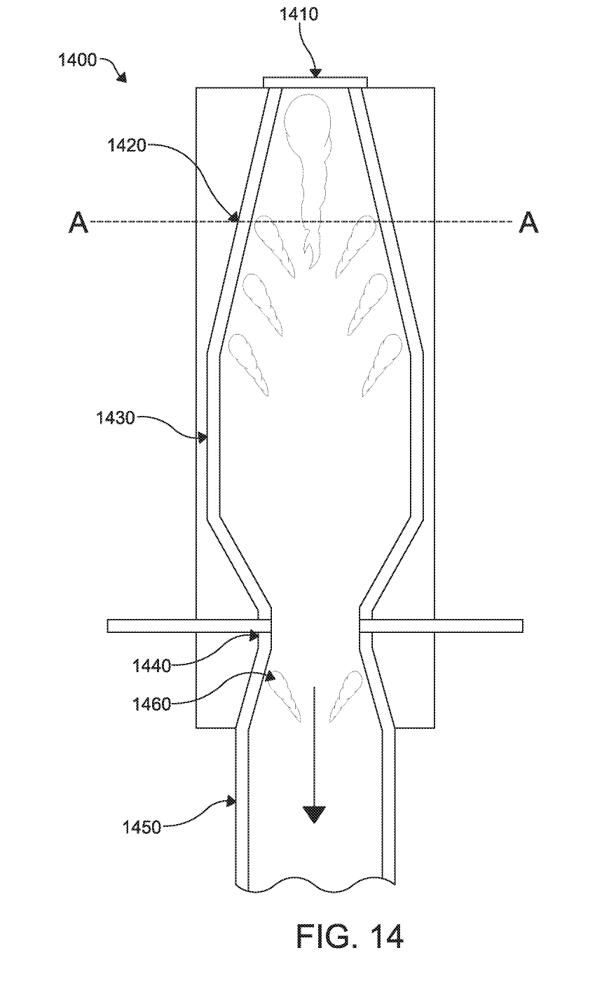

FIG. 14 illustrates another example chamber 1400 herein having a variable angle defining one or more of the chamber 1400 sections. Chamber 1400 can include a first section 1410, a second section 1420, a third section 1430, and feed section 1440 operatively coupled with output 1450. As illustrated, it is shown that first section 1410 can have a conical contour and that the angle of first section 1410 with respect to, e.g., an arbitrary axis, lateral axis, or second section 1420 can vary to make the conical arrangement wider or narrower. As illustrated, the feed section 1440 or throat has a diameter that is less than the adjacent third section 1430 and output 1450. At the feed section 1440, a feedstock may be introduced into the chamber 1400 to affect a reaction process, for example to produce a chemical product using the thermal energy in the chamber 1400. One or more jets 1460 may be disposed downstream (e.g., away from the first section 1410) from the feed section 1440, for example, to introduce a flame or cracking gas downstream the introduction of the feedstock. The one or more jets 1460 may be configured as a stage of combustors or burners and may include any number or arrangement of jets. As shown, the jets 1460 are radial jets. The jets 1460 may have various introduction angles and may be operated to produce various thermal properties.

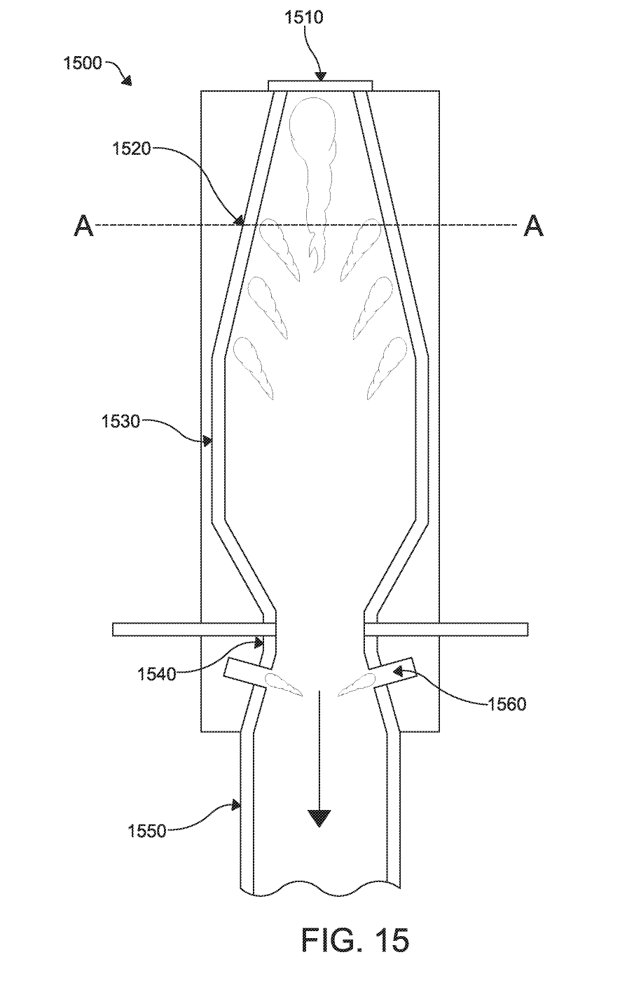

FIG. 15 illustrates another example chamber 1500 herein having a variable angle defining one or more of the chamber 1500 sections. Chamber 1500 can include a first section 1510, a second section 1520, a third section 1530, and feed section 1540 operatively coupled with output 1550. As illustrated, it is shown that first section 1510 can have a conical contour and that the angle of first section 1510 with respect to, e.g., an arbitrary axis, lateral axis, or second section 1520 can vary to make the conical arrangement wider or narrower. As illustrated, the feed section 1540 or throat has a diameter that is less than the adjacent third section 1530 and output 1550. At the feed section 1540, a feedstock may be introduced into the chamber 1500 to affect a reaction process, for example to produce a chemical product using the thermal energy in the chamber 1500. One or more recessed jets 1560 may be disposed downstream (e.g., away from the first section 1510) from the feed section 1540, for example, to introduce flame or cracking gas downstream the introduction of the feedstock. The one or more jets 1560 may be configured as a stage of combustors or burners and may include any number or arrangement of jets. As shown, the jets 1560 are recessed radial jets. The jets 1560 may have various introduction angles and may be operated to produce various thermal properties.

### Aspects

The disclosed systems and methods include at least the following aspects.

Aspect 1. A system, comprising: a combustion chamber having a chamber structure including sidewalls; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and a process feed for providing a feedstock acted upon by the combustion within the combustion chamber, wherein a firing rate of about 30 MMBtu/h to about 1000 MMBtu/h is exhibited in the combustion chamber.

Aspect 2. A system, consisting essentially of: a combustion chamber having a chamber structure including sidewalls; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and a process feed for providing a feedstock acted upon by the combustion within the combustion chamber, wherein a firing rate of about 30 MMBtu/h to about 1000 MMBtu/h is exhibited in the combustion chamber.

Aspect 3. A system, consisting of: a combustion chamber having a chamber structure including sidewalls; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and a process feed for providing a feedstock acted upon by the combustion within the combustion chamber, wherein a firing rate of about 30 MMBtu/h to about 1000 MMBtu/h is exhibited in the combustion chamber.

Aspect 4. The system of any one of aspects 1-3, further comprising: a first sidewall section of the sidewalls having a first cross-section; and a second sidewall section of the sidewalls having a second cross-section, the first cross-section tapering from a chamber cap to the second cross-section.

Aspect 5. The system of aspect 4, wherein the first cross-section comprises a non-constant taper.

Aspect 6. The system of aspect 4, further comprising a second sidewall section having a second cross-section different from the first cross-section and the second cross-section.

Aspect 7. The system of aspect 4, wherein the first stage is arranged through the first cross-section and the second stage is arranged through the second cross-section.

Aspect 8. The system of any one of aspects 1-7, wherein at least one of the one or more first inlets or the one or more second inlets is a mixed gas inlet.

Aspect 9. The system of any one of aspects 1-8, wherein the fluid provided through the one or more first inlets or the one or more second inlets comprises H₂, CO, CH₄, C₂H₆, C₃14₈, or a combination thereof

Aspect 10. The system of any one of aspects 1-9, wherein one or more of the first stage and the second stage is configured operate as a swirl burner.

Aspect 11. The system of any one of aspect 1-10, further comprising a third stage having one or more third inlets, the one or more third inlets having one or more third inlet directions incident to respective areas of the sidewalls at one or more third inlet angles, the one or more third inlets configured to provide fluid for combustion in the combustion chamber, the third stage producing a radial jet within the combustion chamber.

Aspect 12. The system of any one of aspect 1-11, wherein the feedstock comprises hydrocarbon feedstock and acetylene is produced via pyrolysis of the hydrocarbon feedstock by contacting exhaust gases produced from combustion chamber.

Aspect 13. The system of any one of aspect 12, wherein the hydrocarbon feedstock comprises natural gas, methane, paraffinic hydrocarbons, olefinic hydrocarbons, or alcohols, or a combination thereof.

Aspect 14. The system of any one of aspects 1-13, further comprising a cooling structure around at least a portion of the chamber.

Aspect 15. A method for producing a chemical, comprising: firing a first jet within a combustion chamber at a first jet angle, the first jet angle defined by one or more first inlets through a sidewall of the combustion chamber, the first jet is fired by providing fluid through the one or more first inlets, the first jet is an axial jet; and firing two or more second jets within a chamber at two or more second jet angles, the two or more second jet angles defined by two or more second inlets through the sidewall of the combustion chamber, the two or more second jets are fired by providing fluid through the one or more second inlets, the two or more second jets are radial jets; and providing feedstock into the combustion chamber during combustion through a process feed, the feedstock is processed into at least a portion of a product output from the combustion chamber, wherein a firing rate of about 30 MMBtu/h to about 1000 MMBtu/h is exhibited in the combustion chamber.

Aspect 16. A method for producing a chemical, consisting essentially of: firing a first jet within a combustion chamber at a first jet angle, the first jet angle defined by one or more first inlets through a sidewall of the combustion chamber, the first jet is fired by providing fluid through the one or more first inlets, the first jet is an axial jet; and firing two or more second jets within a chamber at two or more second jet angles, the two or more second jet angles defined by two or more second inlets through the sidewall of the combustion chamber, the two or more second jets are fired by providing fluid through the one or more second inlets, the two or more second jets are radial jets; and providing feedstock into the combustion chamber during combustion through a process feed, the feedstock is processed into at least a portion of a product output from the combustion chamber, wherein a firing rate of about 30 MMBtu/h to about 1000 MMBtu/h is exhibited in the combustion chamber.

Aspect 17. A method for producing a chemical, consisting of: firing a first jet within a combustion chamber at a first jet angle, the first jet angle defined by one or more first inlets through a sidewall of the combustion chamber, the first jet is fired by providing fluid through the one or more first inlets, the first jet is an axial jet; and firing two or more second jets within a chamber at two or more second jet angles, the two or more second jet angles defined by two or more second inlets through the sidewall of the combustion chamber, the two or more second jets are fired by providing fluid through the one or more second inlets, the two or more second jets are radial jets; and providing feedstock into the combustion chamber during combustion through a process feed, the feedstock is processed into at least a portion of a product output from the combustion chamber, wherein a firing rate of about 30 MMBtu/h to about 1000 MMBtu/h is exhibited in the combustion chamber.

Aspect 18. The method of any one of aspects 15-17, wherein the combustible fluid provided through the one or more first inlets and/or the one or more second inlets comprises H₂, CO, CH₄, C₂H₆, C₃H₈, or a combination thereof.

Aspect 19. The method of any one of aspects 15-18, wherein the first jet or the two or more second jets is/are configured to operate as a swirl burner.

Aspect 20. The method of any one of aspects 15-19, wherein the two or more second jet angles are non-orthogonal relative to a longitudinal axis of the combustion chamber.

Aspect 21. The method of any one of aspects 15-20, wherein the feedstock comprises hydrocarbon feedstock and acetylene is produced via pyrolysis of the hydrocarbon feedstock by contacting exhaust gases produced from combustion chamber.

Aspect 22. The method of aspect 21, wherein the hydrocarbon feedstock comprises natural gas, methane, paraffinic hydrocarbons, olefinic hydrocarbons, or alcohols, or a combination thereof.

Aspect 23. The method of any one of aspects 15-21, further comprising cooling a sidewall of the combustion chamber.

Aspect 24. A system, comprising: a combustion chamber having a chamber structure including sidewalls, the sidewalls having at least a first section and a second section, the first section having a varying first cross-section and the second section having a second cross-section; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; a process feed for providing a feedstock acted upon by the combustion within the combustion chamber; an output for providing product based at least in part on the chemical; and a cooling structure about at least a portion of the chamber, wherein a firing rate of about 30 MMBtu/h to about 1000 MMBtu/h is exhibited in the combustion chamber.

Aspect 25. A system, consisting essentially of: a combustion chamber having a chamber structure including sidewalls, the sidewalls having at least a first section and a second section, the first section having a varying first cross-section and the second section having a second cross-section; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; a process feed for providing a feedstock acted upon by the combustion within the combustion chamber; an output for providing product based at least in part on the chemical; and a cooling structure about at least a portion of the chamber, wherein a firing rate of about 30 MMBtu/h to about 1000 MMBtu/h is exhibited in the combustion chamber.

Aspect 26. A system, consisting of: a combustion chamber having a chamber structure including sidewalls, the sidewalls having at least a first section and a second section, the first section having a varying first cross-section and the second section having a second cross-section; a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber; a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; a process feed for providing a feedstock acted upon by the combustion within the combustion chamber; an output for providing product based at least in part on the chemical; and a cooling structure about at least a portion of the chamber, wherein a firing rate of about 30 MMBtu/h to about 1000 MMBtu/h is exhibited in the combustion chamber.

In various other aspects, a method can include combining a combustor and a pyrolysis unit to produce chemical intermediates for producing commodity chemicals. A staged combustion chamber pyrolysis reactor can include a mixing section where feedstock is injected to such ends.

Aspects of chambers herein can be used to provide firing rates of 10 MMBtu, 30 MMBtu, values in between, and values above such, in a single chamber. In aspects, firing rates over 600 MMBtu, 1000 MMBtu, ranges there between, or more can be accomplished in a single chamber disclosed herein. Example firing rates and other values are provided for example purposes only and are regarded to include the ranges between any discrete values provided. These firing rates can, in aspects, be accomplished using oxy-natural gas flames, oxy-methane flames, or other oxy-fuels. Fuels can be single gas or mixtures of gas, including gases such as H₂, CO, CH₄, C₂H₆, C₃H₈, and/or other hydrocarbons. Temperature uniformity can be accomplished using staged flames, and particularly one or more radial flames (e.g., swirl flames) in conjunction with an axial flame, which can improve production quality, production efficiency, and system lifecycle.

In an aspect, acetylene is produced via pyrolysis of natural gas by contacting exhaust gases produced from a staged combustion chamber having a mixing section. Gases can include natural gas, methane, and/or paraffinic hydrocarbons such as ethane, propane, butane, and/or hexane, alone or in mixed combinations. In alternative or complementary aspects, olefinic hydrocarbons such as ethene, propene, butene, pentene, and/or hexene can be used, alone or in combination with other gases described. In alternative or complementary aspects, alcohols such as methanol, ethanol, propanol, utenol, pentanol, hexanol, and/or amyl alcohol can be used, alone or in combination with other gases described. In aspects, all of the above can be used in varying combinations.

In an aspect, a system is disclosed comprising a chamber having a chamber structure including sidewalls, a first stage having one or more first inlets, in which the one or more first inlets having one or more first inlet directions incident to respective areas of the sidewalls at one or more first inlet angles. There is also at least a first subsequent stage having one or more subsequent inlets, in which the one or more subsequent inlets having one or more subsequent inlet directions incident to respective areas of the sidewalls at one or more subsequent inlet angles.

In a further aspect of the above, there is included first sidewall section of the sidewalls having a first cross-section and a second sidewall section of the sidewalls having a second cross-section, the first cross-section tapering from a chamber cap to the second cross-section. In a further aspect, the first cross-section includes a non-constant taper. In an alternative or complementary further aspect, the system further includes a subsequent sidewall section having a subsequent cross-section different from the first cross-section and the second cross-section. In an alternative or complementary further aspect, the first stage is arranged through the first cross-section and the subsequent stage arranged through the second cross-section.

In a further aspect, at least one of the one or more first inlets or the one or more subsequent inlets is a mixed gas inlet. In still a further aspect, the system includes at least one subsequent stage. In an alternative or complementary future aspect, the at least one subsequent stage has a different number of inlets than the one or more subsequent inlets.

In a further aspect, two or more of the one or more first inlet directions and the one or more subsequent inlet directions being different. In a further aspect, two or more of the one or more first inlet directions are different and/or two or more of the one or more subsequent inlets are different. In a further aspect, the system additionally includes a cooling structure around at least a portion of the chamber. In still a further aspect, at least one of the one or more first inlets and the one or more subsequent inlets includes a nonlinear bore curving through the sidewalls.

In aspects, there can also be a method for producing a chemical comprising firing a first jet within a chamber at a first jet angle and firing two or more subsequent jets within a chamber at two or more subsequent jet angles. In a further aspect, the method can include cooling at least a portion of the chamber using a cooling structure.

In a further aspect, the chamber includes sidewalls having two or more sidewall sections, the two or more sidewall sections having two or more cross-sections. In a further aspect, at least one of the two or more cross-sections is curved. In another further aspect, the first jet corresponds to a first section of the two or more sidewall sections, and the subsequent jets correspond to subsequent sections of the two or more sidewall sections.

In a further aspect, the first jet is produced using first inlets having first inlet directions, and the subsequent jets produced using subsequent inlets having subsequent inlet directions. In a still further aspect, at least one of the first inlet directions or the subsequent inlet directions is curved.

Additional aspects herein include a system comprising a chamber having a chamber structure including sidewalls. The sidewalls can have two or more sections of varying cross-section. The system also includes a first stage having first inlets which have first inlet directions incident to respective areas of the sidewalls at first inlet angles. There are also two or more subsequent stages having subsequent inlets which have subsequent inlet directions incident to respective areas of the sidewalls at subsequent inlet angles. The subsequent inlet directions vary by stage. There is also included a cooling structure about at least a portion of the chamber.

In the specification and claims, reference is made to a number of terms described hereafter. The singular forms “a,” “an,” and “the” include plural referents unless the context clearly dictates otherwise. Approximating language, as used herein throughout the specification and claims, may be applied to modify a quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Moreover, unless specifically stated otherwise, a use of the terms “first,” “second,” etc., do not denote an order or importance, but rather the terms “first,” “second,” etc., are used to distinguish one element from another.

As used herein, the terms “may,” “may be,” “can,” and/or “can be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As utilized herein, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

To the extent that the term “includes” is used in either the detailed description or the claims, such term is intended to be inclusive in a manner similar to the term “comprising” as “comprising” is interpreted when employed as a transitional word in a claim.

It is to be understood that the terminology used herein is for the purpose of describing particular aspects only and is not intended to be limiting. As used in the specification and in the aspects, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the aspects which follow, reference will be made to a number of terms which shall be defined herein.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular aspects of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or steps.

1. A system, comprising:
a combustion chamber having a chamber structure including sidewalls;
a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber;
a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber; and
a process feed for providing a feedstock acted upon by the combustion within the combustion chamber, wherein a firing rate of about 30 MMBtu/h to about 1000 MMBtu/h is exhibited in the combustion chamber.

2. The system of claim 1, further comprising:
a first sidewall section of the sidewalls having a first cross-section; and
a second sidewall section of the sidewalls having a second cross-section, the first cross-section tapering from a chamber cap to the second cross-section.

3. The system of claim 2, wherein the first cross-section comprises a non-constant taper.

4. The system of claim 2, further comprising a second sidewall section having a second cross-section different from the first cross-section and the second cross-section.

5. The system of claim 2, wherein the first stage is arranged through the first cross-section and the second stage is arranged through the second cross-section.

6. The system of claim 1, wherein at least one of the one or more first inlets or the one or more second inlets is a mixed gas inlet.

7. The system of claim 1, wherein the fluid provided through the one or more first inlets or the one or more second inlets comprises H₂, CO, CH₄, C₂H₆, C₃H₈, or a combination thereof.

8. The system of claim 1, wherein one or more of the first stage and the second stage is configured operate as a swirl burner.

9. The system of claim 1, further comprising a third stage having one or more third inlets, the one or more third inlets having one or more third inlet directions incident to respective areas of the sidewalls at one or more third inlet angles, the one or more third inlets configured to provide fluid for combustion in the combustion chamber, the third stage producing a radial jet within the combustion chamber.

10. The system of claim 1, wherein the feedstock comprises hydrocarbon feedstock and acetylene is produced via pyrolysis of the hydrocarbon feedstock by contacting exhaust gases produced from combustion chamber.

11. The system of claim 10, wherein the hydrocarbon feedstock comprises natural gas, methane, paraffinic hydrocarbons, olefinic hydrocarbons, or alcohols, or a combination thereof.

12. The system of claim 1, further comprising a cooling structure around at least a portion of the chamber.

13. A method for producing a chemical, comprising:
firing a first jet within a combustion chamber at a first jet angle, the first jet angle defined by one or more first inlets through a sidewall of the combustion chamber, the first jet is fired by providing fluid through the one or more first inlets, the first jet is an axial jet; and
firing two or more second jets within a chamber at two or more second jet angles, the two or more second jet angles defined by two or more second inlets through the sidewall of the combustion chamber, the two or more second jets are fired by providing fluid through the one or more second inlets, the two or more second jets are radial jets; and
providing feedstock into the combustion chamber during combustion through a process feed, the feedstock is processed into at least a portion of a product output from the combustion chamber, wherein a firing rate of about 30 MMBtu/h to about 1000 MMBtu/h is exhibited in the combustion chamber.

14. The method of claim 13, wherein the fluid provided through the one or more first inlets or the one or more second inlets comprises H₂, CO, CH₄, C₂HH₆, C₃H₈, or a combination thereof.

15. The method of claim 13, wherein the first jet or the two or more second jets are configured to operate as a swirl burner.

16. The method of claim 13, wherein the two or more second jet angles are non-orthogonal relative to a longitudinal axis of the combustion chamber.

17. The method of claim 13, wherein the feedstock comprises hydrocarbon feedstock and acetylene is produced via pyrolysis of the hydrocarbon feedstock by contacting exhaust gases produced from combustion chamber.

18. The method of claim 17, wherein the hydrocarbon feedstock comprises natural gas, methane, paraffinic hydrocarbons, olefinic hydrocarbons, or alcohols, or a combination thereof.

19. The method of claim 13, further comprising cooling a sidewall of the combustion chamber.

20. A system, comprising:
a combustion chamber having a chamber structure including sidewalls, the sidewalls having at least a first section and a second section, the first section having a varying first cross-section and the second section having a second cross-section;
a first stage having one or more first inlets, the one or more first inlets having one or more first inlet directions incident to respective areas of the sidewalls at one or more first inlet angles, the one or more first inlets configured to provide fluid for combustion in the combustion chamber, the first stage producing one or more of an axial jet and a radial jet within the combustion chamber;
a second stage having one or more second inlets, the one or more second inlets having one or more second inlet directions incident to respective areas of the sidewalls at one or more second inlet angles, the one or more second inlets configured to provide fluid for combustion in the combustion chamber, the second stage producing a radial jet within the combustion chamber;
a process feed for providing a feedstock acted upon by the combustion within the combustion chamber;
an output for providing product based at least in part on the fluid; and
a cooling structure about at least a portion of the chamber, wherein a firing rate of about 30 MMBtu/h to about 1000 MMBtu/h is exhibited in the combustion chamber.
